(19) United States
(12) Patent Application Publication
(10) Pub. No.: US 2004/0047829 A1
(43) Pub. Date: Mar. 11, 2004

(54) Fluids for sanitizing the teats of dairy animals

(76) Inventor: William E. Hahn, Aurora, CO (US)

Correspondence Address: Jonathan J. Wainer, 4 Newman Circle, Natick, MA, 01760, US

(21) Appl. No.: 10/235,460

(22) Filed: Sep. 5, 2002

(51) Int. Cl. A61K 7/075; A61K 7/08

(52) U.S. Cl. 424/70.28

(57) ABSTRACT

The invention provides aqueous sanitizing fluids that include alkyl dimethyl benzyl ammonium chloride (ADBAC) and a skin conditioner. When used as a pre-milking or post-milking sanitizing fluid for sanitizing the teats of milking animals, a dye is also included as an indicator of treatment. The skin conditioner preferably includes a moisturizer, and, for post-milking sanitizing fluids, the fluid may further include a skin growth enhancer, antioxidants, and a viscosity enhancer.

### FIELD OF THE INVENTION

The present invention relates generally to methods and formulations for sanitizing the teats of dairy cows and other milk-producing mammals.

### BACKGROUND OF THE INVENTION

In modem dairy operations the teats and lower portion of the udder are typically sanitized by spraying or dipping teats in a sanitizing fluid, followed some seconds or minutes later by wiping the teats with a cloth or paper towel. These steps are specified in the Grade A Pasteurization Ordinance, U.S. Department of Health and Human Services. After milking is completed, teats are commonly sprayed or dipped with a sanitizing fluid containing compounds to help maintain skin moisture. This step is performed to help reduce the frequency of mastitis infection, which is more likely to occur during a period when the teat orifice remains open.

A variety of compounds have been used as the sanitizing or antimicrobial agent in pre and post milking sprays or dips. Most commonly iodine is used, typically at concentrations of 0.5-1.0%. The iodine is not all in free form but is released from an iodophor. Other sanitizing fluids contain antimicrobial compounds, such as chlorohexidine, dodecyl benzene sulfonic acid, quaternary ammonium, lactic acid, phenol, caprylic-caproic acids, sodium chlorite, hypochlorous acid, and hydrogen peroxide. For the most part these compounds kill microorganisms as a consequence of being strong oxidants or by disruption of the plasma membrane causing loss of control over permeability and consequent death of the cell.

Because iodine is an oxidant, it can irritate the cow's teats and udder. In addition, the iodine can also be irritating to the skin, and can cause drying and cracking. To help alleviate these conditions, the antimicrobial compounds are often mixed with agents that moisturize and help maintain healthy skin condition, such as lanolin and glycerin.

Using iodine as the antimicrobial agent also raises environmental and health concerns. The drippings of iodine solution in the dairy barn are washed out with other waste. These wastes are often held for a time on the farm and leach into the soil and into the ground water. The iodine can then find its way into private wells or even public drinking water. This is undesirable because excess iodine in the diet from drinking water can lead to thyroid problems.

The use of iodine can also be unhealthy for dairy workers, who inhale the iodine spray or dry particles carrying iodine. The excess iodine can be the cause of thyroiditis.

### SUMMARY

The invention, in one aspect, provides an aqueous sanitizing fluid for sanitizing skin. The sanitizing fluid includes alkyl dimethyl benzyl ammonium chloride (ADBAC) in a concentration greater or equal to about 50 mg/L, and a skin conditioner. The sanitizing fluid can further include a dye, such as oxzazine (basic blue dye no. 3), when used for sanitizing the teats of dairy animals. The skin conditioner preferably includes at least a moisturizer, such as sorbitol, glycerin, or hydroxyethyl cellulose. Other skin conditioning compositions that may be used are skin growth enhancers, such as allantoin, and antioxidants, such as ascorbic acid and thioctic acid. The fluid, when used for a post-milking teat dip, can advantageously include a viscosity enhancer, such as hydroxyethyl cellulose. The fluid may also include urea, which is a carrier for ADBAC.

In another aspect, the invention provides an aqueous pre-milking or post-milking teat sanitizing fluid, including ADBAC in a concentration greater or equal to about 200 mg/L, and a dye, such as oxzazine, used as an indicator of which teats have been treated. The formulations may also include a skin conditioner. The skin conditioner preferably includes a moisturizer, such as glycerin or sorbitol, in concentrations of about 10-100 ml/L., to promote good skin condition. The skin conditioner may also include at least one of a skin growth enhancer and an antioxidant.

In yet another aspect, the invention provides an aqueous post-milking sanitizing teat dip, including ADBAC in a concentration of at least about 200 mg/L, and a viscosity enhancer, such as hydroxyethyl cellulose. The dip preferably also includes a dye and a skin conditioner, which may include at least one or more of a moisturizer, a skin growth enhancer, and an antioxidant.

In still another aspect, the invention provides an aqueous sanitizing and hand cleaning fluid that includes ADBAC in a concentration in a range between about 50-200 mg/L, a skin conditioner, and sodium pyrollidone carboxylic acid in a concentration of about 5-40g/L. The skin conditioner preferably includes a moisturizer, such as sorbitol in a concentration of about 40-150 g/L.

Water used in these formulations is preferably softened and filter-sterilized. Preferably, the pH is adjusted to between about 7.0 and 8.0 by the addition of a base, such as sodium hydroxide or potassium hydroxide.

The ADBAC in these sanitizing fluids when used for sanitizing milking animals is preferably in a range of about 200-3000mg/L, and more preferably in a range of about 300-1200 mg/L. Other ingredients in these formulations are selected so as not to act to diminish the antimicrobial activity of the ADBAC.

The dye oxzazine is preferably used in a concentration of about 50-150 mg/L. Embodiments used for post-milking treatment that include the skin growth enhancer allantoin preferably include that ingredient in a concentration of about 1-4 g/L. Embodiments with antioxidants preferably include ascorbic acid in a concentration of about 2-15 g/L, and thioctic acid in a concentration of about 2-5 g/L. Post-milking formulations with hydroxyethyl cellulose as a viscosity enhancer preferably have that ingredient in a concentration of about 2-12 g/L.

In another aspect, the invention provides methods for sanitizing the teats of milking animal. In a pre-milking method, an aqueous pre-milking sanitizing fluid that includes ADBAC is applied to the animal's teats. Excess fluid is then wiped off after waiting at least 15 seconds. The pre-milking sanitizing fluid preferably includes a dye and a skin conditioner, such as a moisturizer. In a post-milking method, after milking an animal a post-milking sanitizing fluid that includes ADBAC and a dye is applied to the animal's teats. The post-milking sanitizing fluid can be applied by either spraying or dipping the teats. The post-milking sanitizing fluid preferably includes a skin moisturizer, and may also include at least one of a skin growth enhancer and an antioxidant. If applied by dipping, the post-milking sanitizing fluid also preferably includes a viscosity enhancer. The post-milking fluid is not wiped off.

There are several advantages to the invention. The formulations of the invention provide antimicrobial protection to teats of dairy animals using chemistry that is non-oxidative and promotes healthy skin condition. The formulations and methods of the invention do not use iodine, and therefore are healthier for dairy workers and animals, and are environmentally safer.

Other objectives and advantages of the invention will become more apparent from the following detailed description when taken in conjunction with the accompanying drawings.

### DETAILED DESCRIPTION

The invention provides environmentally safe formulations for use in sanitizing dairy animals' teats and udders. These formulations are non-irritating, non-oxidative and promote healthy skin.

The principal antibacterial ingredient in these formulations is alkyl dimethyl benzyl ammonium chloride (ADBAC). ADBAC is effective in killing the bacteria associated with mastitis in milking animals, and is not an oxidant. It also has a slight detergent action and so assists in cleaning dirt off of the animal's teats. Solutions of ADBAC can be stable for over a year. It is inexpensive and is obtained commercially in a convenient dry form under the trade name TIMSEN. In TIMSEN, urea is used as a carrier and a buffer in a weight ratio of about three parts urea to two parts ADBAC by weight. Urea will be in the sanitizing fluid when TIMSEN is used as the stock source for ADBAC, however, it will be understood that urea is not an active ingredient in these formulations.

Aqueous sanitizing fluid formulations according to the invention include ADBAC and a skin conditioner. The ADBAC is preferably in concentrations of at least about 200 mg/L, and more preferably in a concentration of about 400-1200 mg/L. Softened, sterile-filtered water is preferably used as a solvent. If necessary, the pH is adjusted to between about 7.0-8.0 by the addition of a basic agent, such as sodium hydroxide or potassium hydroxide. Preferably, a dye is used as an indicator that an animal has been treated.

The skin conditioner can include one or more of the following: a moisturizer, a skin growth enhancer to promote healing, and an antioxidant. Examples of moisturizers include sorbitol and glycerin. An example of a skin growth enhancer is allantoin. Examples of antioxidants are ascorbic acid and thioctic acid. Embodiments of post-milking sanitizing fluids may also advantageously include a viscosity enhancer, such as hydroxyethyl cellulose, which also has moisturizing properties.

In the examples provided herein, concentrations or amounts refer to ready to use fluid. Although colorizing agents or dyes are shown in the formulations, such agents are non-interactive and, like urea, are not part of the effective ingredients. They may be used as markers to indicate which animals and which parts of the animals have been treated.

Fluid A: Pre- or Post-milking Sanitizing Fluid

| | |
|---|---|
| 1. Alkyl dimethyl benzyl ammonium chloride | 200-1200 mg/L |
| 2. Sorbitol | 10-100 g/L |
| 3. Dye | as needed |

Fluid B: Post-milking Sanitizing Fluid

| | | |
|---|---|---|
| 1. Alkyl dimethyl benzyl ammonium chloride | 200-1200 | mg/L |
| 2. Sorbitol | 10-100 | g/L |
| 3. Allantoin | 1-4 | g/L |
| 4. Ascorbic acid | 2-15 | g/l |
| 5. Thioctic acid | 2-5 | g/L |

| | |
|---|---|
| 6. Dye | as needed |

Fluid C: Post-milking Sanitizing Fluid

| | | |
|---|---|---|
| 1. Alkyl dimethyl benzyl ammonium chloride | 200-1200 | mg/L |
| 2. Sorbitol | 10-100 | g/L |
| 3. Hydroxyethyl cellulose | 2-12 | g/L |

| | |
|---|---|
| 4. Dye | as needed |

Fluid D: Post-milking Sanitizing Fluid

| | | |
|---|---|---|
| 1. Alkyl dimethyl benzyl ammonium chloride | 200-1200 | mg/L |
| 2. Hydroxyethyl cellulose | 2-12 | g/L |

| | |
|---|---|
| 3. Dye | as needed |

Fluid E: Post-milking Sanitizing Fluid

| | | |
|---|---|---|
| 1. Alkyl dimethyl benzyl ammonium chloride | 200-1200 | mg/L |
| 2. Sorbitol | 10-100 | gm/L |
| 3. Hydroxyethyl cellulose | 2-12 | g/L |
| 4. Allantoin | 1-4 | g/L |
| 5. Ascorbic acid | 2-15 | g/L |
| 6. Thioctic acid | 2-5 | g/L |

| | |
|---|---|
| 7. Dye | as needed |

Fluid F. Post-milking Sanitizing Fluid

| | | |
|---|---|---|
| 1. Alkyl dimethyl benzyl ammonium chloride | 200-1200 | mg/L |
| 2. Hydroxyethyl cellulose | 2-12 | g/L |
| 3. Allantoin | 1-4 | g/L |
| 4. Ascorbic acid | 2-15 | g/L |
| 5. Thioctic acid | 2-5 | g/L |

| | |
|---|---|
| 6. Dye | as needed |

In Fluids B, E and F, the addition of allantoin stimulates skin growth and healing. Also in Fluids B, E and F, the antioxidants ascorbic acid and thioctic acid also promote healing and help protect skin from oxidative damage. The post-milking sanitizing fluid may include one, two, or all three of these components, however, the inclusion of all three has been found especially effective. In addition, there are other antioxidants known or to be discovered that may serve as suitable substitutes.

Fluids C, D, E and F include hydroxyethyl cellulose, which is non-interactive. This material, which is also used in dermatological preparations, acts as a lubricant and enhances the viscosity of the sanitizing fluid and so helps hold a protective layer of sanitizer on the teat. After drying a protective film remains until the material flakes off the skin surface. In a preferred embodiment, the hydroxyethyl cellulose is in a range of about 5-8 g/L, and more preferably in a range of 5-7 g/L.

It is noted that in the above examples, sorbitol is used as the sole moisturizer in Fluids A and B, and in Fluids D and F hydroxyethyl cellulose serves that purpose. Fluids C and E include both sorbitol and hydroxyethyl cellulose. Glycerin may also be used as a moisturizing agent. However, glycerin lubricates, so the teat cup may tend to slip off if glycerin were used in a pre-milking sanitizing fluid. A better seal is provided when sorbitol is used in the pre-milking sanitizing fluid.

A pre-milking sanitizing fluid is preferably sprayable and is wiped off quickly. For these reasons it is preferred not to use growth enhancers or antioxidants in such formulations, as they will be of little value when removed quickly. Hydroxyethyl cellulose thickens the fluid, and so its use is preferably reserved for post-milking dips.

Each of the above sanitizing fluids includes a dye as a visual indicator that the animal has been treated. In preferred embodiments, oxzazine dye (basic blue No. 003) is used in amounts of about 50-150 mg/L. Other dyes may also be suitable for use with these sanitizing formulations.

The sanitizing fluids are used in the following manner. Prior to milking, the dairy animals' teats are either sprayed or dipped in a pre-milking sanitizing fluid, such as sanitizing fluid A. The fluid is left on for a period of at least about 15 seconds, and preferably at least about 30-75 seconds, and then excess fluid can be wiped off in the normal manner prior to milking. Some residue remains to continue killing bacteria.

After milking, a post-milking sanitizing fluid is applied to the teats. When using a post-milking sanitizing fluid that does not contain hydroxyethyl cellulose, such as sanitizing fluids A and B, the fluid can be sprayed on the teats. If the sanitizing fluid includes hydroxyethyl cellulose, such as post-milking sanitizing fluids C, D, E or F, then it should be dipped and not sprayed. The post-milking sanitizing fluid should not be wiped off. Rather, it should be left on the teat to provide a protective coating for the teats and particularly for the teat orifices.

Pre- and post-milking formulations with ADBAC concentrations on a range of 200-1200 mg/L have been tested for the ability to kill a broad spectrum of microorganisms from dairy cows, manure, milking barn floors and equipment. In a first, general test, swab samples were recovered in sterile saline and cultured on blood agar microbiological plates. These microorganisms were not identified by genus or species. Included among the colonies were various soil bacteria, molds and yeasts. Six bacterial and two mold colonies were isolated and kill time measurements were performed. All bacterial species were killed within 10-40 seconds of exposure to either the pre or post milking sanitizing fluids. Mold isolates were killed after 75-150 seconds exposure. Killing was determined by culture on blood agar microbiological plates. No growth was observed on these plates.

To further test the antimicrobial properties of the sanitizing fluids of the invention, samples of bacteria from packaged meats were also treated. It is well known that packaged meat can contain bacteria, such as salmonella, that can even be resistant to antibiotics. Samples from absorbent pads of packaged chicken were diluted in sterile saline and aliquots were spread on nutrient agar microbiological plates. Colonies were grown and 10 colonies were selected and tested for sensitivity to the sanitizing fluids. Exposure times ranged from 15 seconds to two minutes. Samples were placed in 100 volumes of sterile saline and aliquots were spread onto blood agar microbiological plates. After 24, 48, and 72 hours of incubation no colonies were observed. As a control, untreated specimens were similarly placed in 100 volumes of sterile saline and aliquots placed on blood agar microbiological plates. Colonies almost too numerous to count were present on these control plates.

In addition, tests of the effectiveness of the sanitizing fluids were conducted with specific, identified microbial species. The species tested were Staphylococcus aurezs, Escherichia coli, Streptococcus agalactica, and Candida albicans. A commercial laboratory performed tests on these species.

Overnight broth cultures of S. aureus, E. coli, and S. agalactia were grown aerobically in tryptic soy broth (TSB) at 37 C. Cultures were centrifuged, rinsed with saline and diluted to give 80% transmittance at wavelength of 530 nm, and the suspension was used as an inoculum. C. albicans was harvested from a Sabouraud Dextrose Agar (SDA) slant, centrifuged, rinsed with saline and diluted to give 5% transmittance at wavelength of 530 nm, and this suspension was also used as an inoculum.

The products were dispensed into a sterile container, inoculated with 0.1 ml of the organism suspension (zero time) and mixed well. After 30 seconds exposure 1.0 ml was transferred to a 9.0 ml DID broth blank. Serial fold dilutions in 9.0 ml sterile saline blanks were prepared and plated in duplicate using SDA for C. Albicans, and Letheen Agar (LA) for S. Aureus, E. coli, and S. agalactia.

SDA palates were incubated at 20-25 C. for 5-7 days until growth was observable on the plates, while LA plates were incubated at 30-35 C. for 3 days, also until growth was observed. The results are shown in Table 1. The numbers indicate colony-forming units recovered per ml, and the reduction (kill) percentage. These results show that no colonies were obtained after brief exposure to sanitizing Fluids 1 and 2 of either the bacterial species or the yeast (Candida). In these tests >99.9=no observed growth; 100% is not used for statistical reasons of sampling. (Fluid 1=600 mg/L ADBAC, 900 mg/L urea, 20 ml/L glycerin, and 50 mg/L oxzazine dye; Fluid 2=600 mg/L ADBAC, 900 mg/L urea, 20 ml/L sorbitol, and 50 mg/L oxzazine dye.)

TABLE 1
Organism Counts after 30 seconds and after 2 minutes.

| | Control | Fluid 1 | Fluid 1 | Fluid 2 | Fluid 2 |
|---|---|---|---|---|---|
| Organism | Counts | Count > 30 s | Count > 2 m | Count > 30 s | Count > 2 m |
| E. coli | 3.6 × 10⁵ | <10 | 1.0 × 10² | <10 | <10 |
| #8739 | | >99.9 | >99.9 | >99.9 | >99.9 |
| % reduction | | | | | |
| S. aureus | 8.96 × 10⁵ | <10 | 1.0 × 10² | 1.0 × 10² | <10 |
| #6538 | | >99.9 | >99.9 | >99.9 | >99.9 |
| % reduction | | | | | |
| S agalactia | 4.3 × 10⁴ | <10 | 1.5 × 10² | <10 | <10 |
| #624 | | >99.9 | >99.6 | >99.9 | >99.9 |
| % reduction | | | | | |
| C albicans | 1.4 × 10⁵ | <10 | <10 | <10 | <10 |
| #10231 | | >99.9 | >99.9 | >99.9 | >99.9 |
| % reduction | | | | | |

The sanitizing fluids of the invention were also tested with dairy cows to observe the skin condition of the animals after use. In this test, the condition of the teat skin was compared between a dairy where iodine-based sanitizers were in use, and a dairy where sanitizing fluids 1 and 2 had been used for about four months. In general, the overall appearance of the teat skin was noticeably better when fluids 1 and 2 were used in contrast to use of conventional iodine-based fluids. Of particular interest was the appearance of the teat orifices. The health of the skin in this area is of particular concern for dairies. When the teat skin is pliable and free of cracks and chapping, the orifice skin is smooth in contour. When the skin is in poor condition the orifice may be folded or creased yielding a “star” like appearance.

Extensive examination of teat orifices of cows treated with fluids 1 and 2 revealed that these fluids were not destructive to teat skin, permitting overall maintenance of desirable skin conditions. In contrast, teats of cows treated with iodine-based solutions that are highly oxidative, displayed the orifice star and showed other skin irritations much more frequently than cows treated with fluids 1 and 2.

Sanitizing fluid A is compatible with the use of micro fiber wiping cloths. Use of these cloths improves skin cleaning and more surface area is available to trap dirt particles and gently abrade sloughing cells from the skin. The cloths are also very durable. They can survive 2,000 use-wash cycles. Cloths used in our tests were composed of polyamide (20-30%) and polyester (70-80%) and were produced by Advanced Cleaning Technics, of Viskafors, Sweden.

In addition to the above-described embodiments for sanitizing fluids for treating dairy animals, the invention also provides a formula for hand cleaning and sanitization. Milkers are required to keep hands and/or gloves in sanitary condition to avoid spreading infection from one cow to another. Another sanitizing formula according to the invention that was tested and found suitable for hand cleaning is as follows:

Fluid G: Hand-cleaning Sanitizing Fluid

| | | |
|---|---|---|
| 1. Alkyl dimethyl benzyl ammonium chloride | 50-200 | mg/L |
| 2. Sodium pyrollidone carboxylic acid | 5-40 | g/L |
| 3. Sorbitol | 40-150 | g/L |

The hand solution can be used by applying 1-3 ml into the palm of the hand, and then rubbing the hands together for about 10-15 seconds. Hands can be let to air dry or wiped with a clean cloth, a paper towel, or another suitable wipe. The solution can also be used to wet a suitable paper or fiber cloth wipe that can be packaged in foil or plastic, which will provide a cleaning and sanitizing tool that can be carried conveniently for personal use.

All of the references cited herein, including patents, patent applications, and publications, are hereby incorporated in their entireties by reference.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An aqueous sanitizing fluid for sanitizing skin, comprising:
alkyl dimethyl benzyl ammonium chloride (ADBAC) in a concentration greater or equal to about 50 mg/L; and
a skin conditioner.

2. The sanitizing fluid of claim 1, having a pH in the range of about 7-8.

3. The sanitizing fluid of claim 1, further comprising a dye.

4. The sanitizing fluid of claim 3, wherein the concentration of ADBAC is greater or equal to about 200 mg/L.

5. The sanitizing fluid of claim 3, wherein the skin conditioner comprises a moisturizer.

6. The sanitizing fluid of claim 5, wherein the moisturizer comprises sorbitol.

7. The sanitizing fluid of claim 5, wherein the moisturizer comprises glycerin.

8. The sanitizing fluid of claim 5, wherein the moisturizer comprises hydroxyethyl cellulose.

9. The sanitizing fluid of claim 5, further comprising a viscosity enhancer.

10. The sanitizing fluid of claim 5, wherein the skin conditioner further comprises at least one of a skin growth enhancer and an antioxidant.

11. The sanitizing fluid of claim 10, further comprising a viscosity enhancer.

12. The sanitizing fluid of claim 1, further comprising urea.

13. The sanitizing fluid of claim 4, wherein the ADBAC is in a concentration of up to about 3000 mg/L.

14. An aqueous pre-milking or post-milking teat sanitizing fluid, comprising:
alkyl dimethyl benzyl ammonium chloride (ADBAC) in a concentration greater or equal to about 200 mg/L; and
a dye.

15. The sanitizing fluid of claim 14, further comprising a skin conditioner.

16. The sanitizing fluid of claim 15, wherein the skin conditioner comprises a moisturizer.

17. The sanitizing fluid of claim 16, wherein the moisturizer comprises sorbitol.

18. The sanitizing fluid of claim 17, wherein the sorbitol is in a concentration of at least about 10 ml/L.

19. The sanitizing fluid of claim 16, wherein the skin conditioner further comprises at least one of a skin growth enhancer and an antioxidant.

20. An aqueous post-milking sanitizing teat dip, comprising:
alkyl dimethyl benzyl ammonium chloride (ADBAC) in a concentration greater or equal to about 200 mg/L; and
a viscosity enhancer.

21. The sanitizing teat dip of claim 20, wherein the viscosity enhancer comprises hydroxyethyl cellulose.

22. The sanitizing teat dip of claim 20, further comprising a dye.

23. The sanitizing teat dip of claim 22, wherein the dye comprises oxzazine dye.

24. The sanitizing teat dip of claim 20, further comprising a skin conditioner.

25. The sanitizing teat dip of claim 24, wherein the skin conditioner comprises at least one of a moisturizer, a skin growth enhancer and an antioxidant.

26. The sanitizing teat dip of claim 25, wherein the moisturizer comprises sorbitol.

27. The sanitizing teat dip of claim 24, wherein the skin growth enhancer comprises allantoin.

28. The sanitizing teat dip of claim 24, wherein the antioxidant comprises at least one of ascorbic acid and thioctic acid.

29. The sanitizing teat dip of claim 24, further comprising a dye.

30. An aqueous sanitizing fluid for sanitizing the teats of milking animals, comprising:
alkyl dimethyl benzyl ammonium chloride (ADBAC) in a concentration of at least about 200 mg/L;
sorbitol in a concentration of at least about 10 g/L; and
a dye.

31. The sanitizing fluid of claim 30, wherein the dye comprises oxzazine dye in a concentration of at least about 50 mg/L.

32. The sanitizing fluid of claim 30, further comprising hydroxyethyl cellulose.

33. The sanitizing fluid of claim 32, wherein the hydroxyethyl cellulose is in a concentration of at least 2 g/L.

34. The sanitizing fluid of claim 32, further comprising a skin growth enhancer.

35. The sanitizing fluid of claim 34, wherein the skin growth enhancer includes allantoin.

36. The sanitizing fluid of claim 32, further comprising an antioxidant.

37. The sanitizing fluid of claim 36, wherein the antioxidant includes at least one of ascorbic acid and thioctic acid.

38. The sanitizing fluid of claim 30, further comprising a skin growth enhancer.

39. The sanitizing fluid of claim 38, wherein the skin growth enhancer includes allantoin.

40. The sanitizing fluid of claim 30, further comprising an antioxidant.

41. The sanitizing fluid of claim 40, wherein the antioxidant includes at least one of ascorbic acid and thioctic acid.

42. An aqueous sanitizing and hand cleaning fluid, comprising:
alkyl dimethyl benzyl ammonium chloride (ADBAC) in a concentration of about 50-200 mg/L;
a skin conditioner; and
sodium pyrollidone carboxylic acid.

43. The fluid of claim 42, wherein the skin conditioner comprises a moisturizer.

44. The fluid of claim 43, wherein the moisturizer comprises sorbitol in a concentration of about 40-150 g/L, and wherein the sodium pyrollidone carboxylic acid is in a concentration of about 5-40 g/L.

45. A method for sanitizing teats of a milking animal, comprising:
prior to milking, applying an aqueous sanitizing fluid to the teats of the animal, the fluid comprising alkyl dimethyl benzyl ammonium chloride (ADBAC); and
wiping excess fluid off the animal after waiting at least ten seconds.

46. The method of claim 45, wherein the fluid further comprises a dye.

47. The method of claim 46, wherein the fluid further comprises a skin moisturizer.

48. The method of claim 47, wherein the ADBAC is in a concentration of at least about 200 mg/L.

49. A method for sanitizing the teats of a milking animal, comprising:
milking the animal; and
then applying an aqueous sanitizing fluid to the teats of the animal, the fluid comprising alkyl dimethyl benzyl ammonium chloride (ADBAC); and
a dye.

50. The method of claim 49, wherein the fluid further comprises a skin moisturizer.

51. The method of claim 50, wherein the ADBAC is in a concentration of at least about 200 mg/L.

52. The method of claim 50, wherein the fluid further comprises a viscosity enhancer.

53. The method of claim 52, wherein the fluid further comprises a skin moisturizer.

54. The method of claim 52, wherein the fluid further comprises at least one of a skin growth enhancer and an antioxidant.

55. The method of claim 52, wherein applying the fluid included dipping the teats in the fluid.
